(19) United States
(12) Patent Application Publication
(10) Pub. No.: US 2009/0255534 A1
(43) Pub. Date: Oct. 15, 2009

(54) Sealed Vaporization Cartridge and Vaporization Systems for Using

(76) Inventor: Greg Paterno, Scottsdale, AZ (US)

Correspondence Address: ETHERTON LAW GROUP, LLC, 2010 E. University Drive, Suite 25, Tempe, AZ, 85281, US

(21) Appl. No.: 12/422,894

(22) Filed: Apr. 13, 2009

Related U.S. Application Data
(60) Provisional application No. 61/124,114, filed on Apr. 11, 2008.

(51) Int. Cl. A61M 16/12

(52) U.S. Cl. 128/203.21; 128/203.26; 128/203.27

(57) ABSTRACT

A vaporization system for an inhalable substance includes a sealed cartridge. The sealed cartridge contains the chemical solution to be vaporized, a heating element which vaporizes the solution, and a wicking material for retaining the solution and bringing it into contact with the heating element through capillary action. The sealed cartridge may be disposable, having inexpensive elements so the cartridge can be replaced with little cost. Alternatively the sealed cartridge may be fully integrated with other components into a base unit, which may be disposable or may include a refill port which allows a user to refill the sealed cartridge with solution once the contained solution has been fully vaporized. A power source connects to the heating element and is activated by a switch that may be a breath detector. A disposable mouthpiece may be connected to the cartridge. The mouthpiece has an airway through which the vaporized solution is inhaled. The airway is restricted by a trap that prevents unvaporized droplets of the solution from passing through the airway into a user's mouth.

### CROSS-REFERENCE TO RELATED APPLICATIONS

This application claims the benefit of co-pending provisional application No. 61/124,114 filed Apr. 11, 2008.

### FIELD OF INVENTION

This invention relates to inhalation devices. This invention relates particularly to a low-cost electronic vaporization system for an inhalation device that prevents leakage of the substance to be vaporized.

### BACKGROUND

The delivery of chemicals, particularly medicaments and other compounds having a physical or physiological effect on a person, can be accomplished by vaporizing a solution containing the desired elements; the vapor is then inhaled by the user, whereby the desired elements enter the user's blood stream through the lungs. The solution is typically vaporized by bringing it into contact with a heating element. Where the chemical compound is contained in a plant, this delivery method is often preferred to burning the plant because it eliminates the ingestion of smoke and other combustion byproducts, which can have negative health effects and also create a foul taste. Vaporization may also allow ingestion of very small quantities of a chemical compound which is poisonous to humans in larger quantities.

An example of such a compound is nicotine. Nicotine is known to have therapeutic effects in humans, for example as an anti-inflammatory and in treatments for some brain and neurological disorders. However, nicotine is typically ingested through cigarettes or chewing tobacco, which have other harmful, even carcinogenic, chemicals in them. Liquid nicotine does not have these chemicals in it, but it is easily absorbed through the skin. Because it is so poisonous, accidental contact can be fatal. Some existing vaporization systems store liquid nicotine in media which is prone to leakage. Further, some of the unvaporized solution may enter the airway that transports the vapor to the user's mouth, which the user may then suck out of the system into his mouth by inhaling. A nicotine delivery system that eliminates the negative effects of combustion and cigarettes, and also protects the user from leakage or inhalation of the nicotine-containing solution, is desired. Such a system would be equally applicable to other chemical compounds, whether dangerous or not.

Existing vaporization systems designed for personal use suffer from contamination problems. Once a user vaporizes a solution, residue may collect in the system. If the user then desires a different solution, the vapors of the second solution may be contaminated by chemicals or flavors left over from previous uses. A system which allows the user to dispose of potentially contaminated components is desired. Such a system should also be inexpensive, to allow the user to purchase new parts without significant cost.

Therefore, it is an object of this invention to provide a vaporization system that is safer to use than existing systems. It is a further object that the system allow the user to easily and inexpensively eliminate contaminants. Another object of this invention is to provide a sealed vaporization cartridge that limits usage to the solution contained therein and can be constructed and replaced without significant cost.

### SUMMARY OF THE INVENTION

Various embodiments of a novel vaporization system include a sealed cartridge. The sealed cartridge contains the chemical solution to be vaporized, a heating element which vaporizes the solution, and a wicking material for retaining the solution and bringing it into contact with the heating element through capillary action. The solution cannot escape the cartridge in liquid form.

A base unit includes a power source which connects to the heating element, a switch for activating the power source, and an air port which connects to an airway through the sealed cartridge. In one embodiment, the sealed cartridge plugs into the base unit and is unplugged and disposed of when all the solution has been vaporized. The elements of the sealed cartridge are inexpensive so the cartridge can be replaced with little cost. In another embodiment, the sealed cartridge is fully integrated with the base unit, forming a one-piece unit. The one-piece unit may be disposable, or alternatively may include a refill port which allows a user to refill the sealed cartridge with solution once the contained solution has been fully vaporized.

A disposable mouthpiece may be connected to the cartridge. In a one-piece unit the mouthpiece is integrated into the base unit. The mouthpiece has an airway through which the vaporized solution is inhaled. The airway is restricted by a trap which prevents droplets of the solution which are not fully vaporized from passing through the airway into a user's mouth.

### BRIEF DESCRIPTION OF THE DRAWINGS

FIG. 1 is a perspective view of the sealed cartridge of the present invention, with part of the shell cut away to show the vaporizing system.

FIG. 2a is a perspective view of the preferred embodiment of the sealed cartridge with part of the shell cut away to show the vaporizing system.

FIG. 2b is a perspective view of an alternate embodiment of the sealed cartridge with part of the shell cut away to show the vaporizing system.

FIG. 2c is a perspective view of an alternate embodiment of the sealed cartridge with part of the shell cut away to show the vaporizing system.

FIG. 3a is a perspective view of one embodiment of the vaporizing system.

FIG. 3b is a perspective view of another embodiment of the vaporizing system using graphite rods.

FIG. 3c is a side sectional view of another embodiment of the vaporizing system.

FIG. 4 is a perspective view of the sealed cartridge connected to the mouthpiece.

FIG. 5 is a perspective sectional view of an inhalation device.

FIG. 6a is a side view of the section of FIG. 5, showing the switch.

FIG. 6b is an exploded perspective view of an alternate embodiment of the switch.

FIG. 7 is a perspective view of a one-piece inhalation device.

FIG. 8 is a side sectional view of a one-piece inhalation device taken along line A-A of FIG. 7.

FIG. 9 is a perspective view of a sealed cartridge with a recharge port.

### DETAILED DESCRIPTION OF THE INVENTION

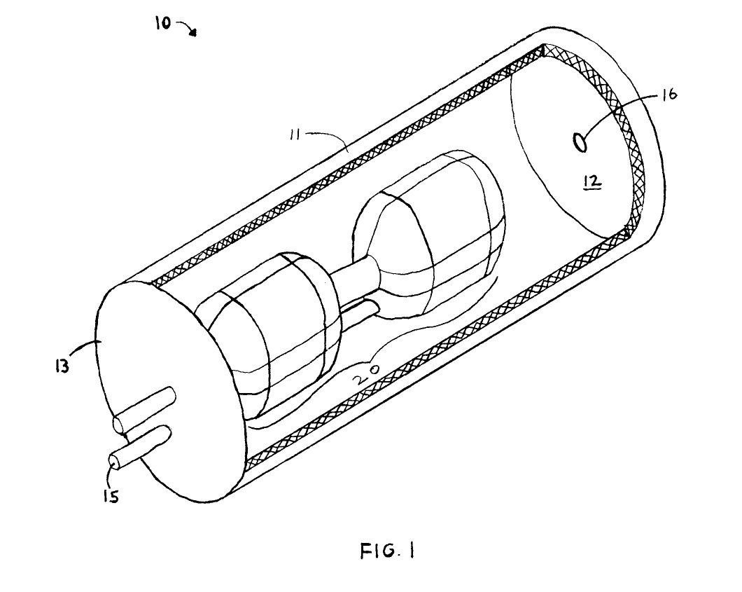

FIG. 1 illustrates a sealed cartridge, designated generally as 10, for containing a solution to be vaporized. The cartridge 10 is insertable into and removable from a compatible inhalation device, allowing for easy replacement when the solution is exhausted or the functionality of the cartridge 10 begins to degrade. The cartridge 10 is sealed to prevent leakage of the solution inside until the cartridge 10 is to be used, allowing for safe handling of the cartridge 10 by a user. A compatible inhalation device, such as those described below and illustrated in the figures, has a power source with terminals that align with the cartridge 10 when it is inserted into the inhalation device, creating an electrical contact that allows the user to activate the device and vaporize the solution in the cartridge 10.

The cartridge 10 has a shell 11 with a base 12 and a cap 13 attached to the shell 11 to create a hollow space therein. The hollow space contains a vaporizing system 20 for storing and vaporizing the solution. The cartridge 10 also comprises at least one air intake aperture 15 and at least one vapor release aperture 16. The cartridge 10 may also comprise a heat-dissipating sleeve 18 in contact with the outer surface of the shell 11.

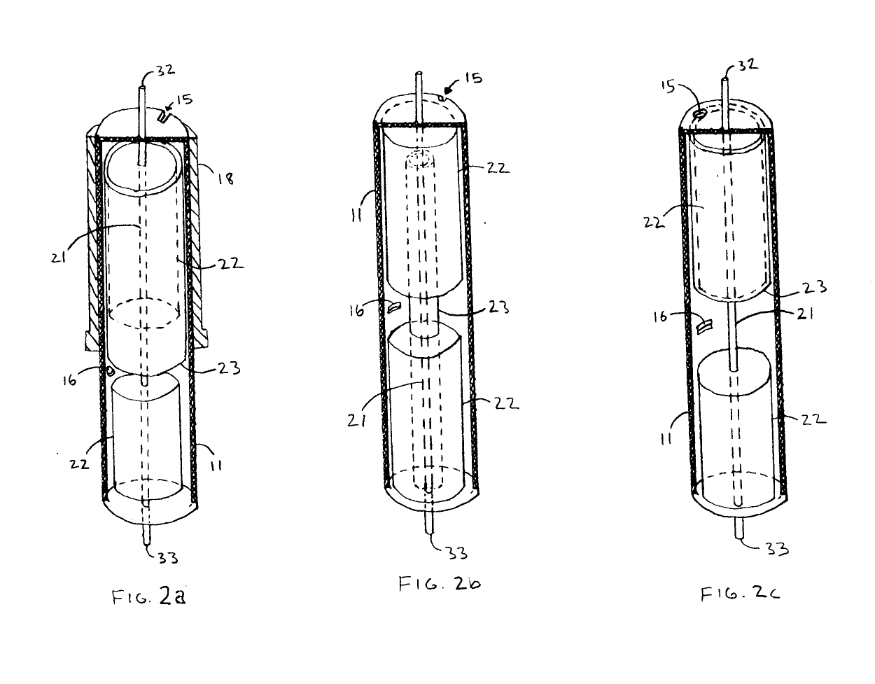

The shell 11 may be a chemically inert, non-porous material, including but not limited to metal, certain polymers, glass, and ceramic. The shell 11 material is chemically inert so as not to alter the solution to be vaporized, and non-porous so as not to absorb the solution in the cartridge 10. The shell 11 material may conduct heat but should be resistant to deformation at typical vaporization temperatures. Preferably, the shell 11 is brass. The base 12 and cap 13 are also non-porous and heat resistant. The cartridge 10 preferably has a round or elliptical cross-section and is most preferably cylindrical. The points of attachment between the shell 11 and the base 12 and cap 13 are watertight and preferably also airtight. The base 12 and cap 13 may be the same material as the shell 11 so that a single mold may cast the three pieces as a single piece, or they may be a different material that can be adhesively attached to the shell 11, such as metal or non-porous plastic, or non-adhesively attached to the shell 11. The apertures 15 and 16 may be integrated into one or more of the shell 11, base 12, and cap 13, or they may be added after the cartridge 10 is sealed with the solution inside. Preferably, the apertures 15 and 16 are cut into the shell 11 and cap 12 in the locations shown in FIG. 2a after the cartridge 10 has been assembled and sealed. The apertures 15 and 16 may then be covered with a material, completely sealing the cartridge 10 until it is ready for use. The covering material may be a thin sheet of plastic or metal, such as aluminum foil. A compatible inhalation device is configured to puncture the covering material when the cartridge 10 is inserted, providing airflow through the cartridge 10. For example, the inhalation device may have protrusions (not shown), such as plastic tabs or small metal spikes, aligned with the apertures 15 and 16 in order to puncture the covering material. Alternatively, the user may manually remove the covering material before inserting the cartridge 10. The vapor release aperture 16 may be slot-shaped, as shown in FIG. 2a, to prevent unvaporized droplets from escaping the sealed cartridge, or may be round as shown in FIG. 2c if unvaporized droplets are not a concern. An additional drop-catching structure (not shown) may be integrated into the vapor release aperture 16.

Referring to FIGS. 2a-c, the vaporization system 20 may comprise a heating element 21, one or more reservoirs 22 for storage and wicking of the solution, and a heat retention structure 23. The heating element 21 may be a metal alloy comprising at least nickel and chromium. Preferably, the heating element 21 is at least one nickel chromium wire which runs through the center of the cartridge 10. The wire may be a single strand or several strands braided together, and may be any gauge that can reach the desired temperature without degrading. Preferably, the heating element 21 comprises three strands of 34-gauge nickel chromium wire, braided loosely to provide additional capillary action for bringing the solution into contact with the heating element. The ends of the heating element 21 serve as the electrical contact points for conducting a current through the heating element 21. Upon insertion of the cartridge 10, the ends align with the terminals of the power source in the inhalation device.

A reservoir 22 is made of a porous media that stores the solution when the heating element 21 is not activated, and transports the solution toward the vaporizing heat by capillary action when the heating element 21 is activated. The reservoir 22 prevents leakage of the solution, in liquid form, through any aperture in the cartridge 10. Preferably, the reservoir 22 is a sheet of nickel foam, cut to fit and then rolled around the heating element 21 to form a cylinder, such that the heating element 21 runs through the reservoir substantially parallel to the reservoir's 22 axis. The reservoir 22 may be a different shape in alternate embodiments, such as a sphere, box, or sheet. There are preferably two reservoirs 22 of nickel foam separated by a short distance, with the heating element 21 spanning the distance so that the vaporization system forms a “dumbbell” shape. This dumbbell shape provides adequate storage for the solution while maximizing the capillary surface area of each reservoir 22 so that the solution may be wicked to the heating element 21 from both ends of each reservoir 22. Nickel foam is available in several densities, which may be combined within the vaporization system 20 to facilitate better storage, wicking, or both. Preferably, the nickel foam has a thickness in the range of about 1.7 to 2.2 millimeters and a density of between 320 and 1450 grams per square meter, with a pore size of between 450 and 800 microns. More preferably, the nickel foam is selected from one of the following groups of thickness, density, and pore size, respectively: 1.7 mm, 320 g/m², and 590 microns; 1.7 mm, 420 g/m², and 450 microns; 1.7 mm, 420 g/m², and 580 microns; 2.2 mm, 800 g/m², and 800 microns; and 1.7 mm, 1450 g/m², and 580 microns. The nickel foam may have the same or different densities in each reservoir 22.

The heat retention structure 23 is a semi-porous, heat-retaining material, such as ceramic, porcelain, or alumina, which encircles all or a portion of the heating element 21 within the cartridge 10. The porous aspect of the material may also be artificially created, such as by perforating the material. The heat retention structure is preferably rigid, semi-porous, unglazed ceramic. When the heating element 21 is activated, the heat retention structure 23 intensifies the heat generated within the heat retention structure 23 while partially insulating the space outside the heat retention structure 23. In the preferred embodiment, shown in FIG. 2a, the heat retention structure 23 is a tube which encircles a portion of the heating element 21, one of the reservoirs 22, and the gap between the reservoirs 22. In an alternate embodiment, shown in FIG. 2b, the heat retention structure 23 is a tube that encircles only the heating element 21, separating it from the reservoirs 22. In another alternate embodiment, shown in FIG. 2c, the heat retention structure 23 is a tube which encircles part of the heating element 21 and one of the reservoirs 22, but does not enclose the gap between the reservoirs 22 so that the heat generated by the part of the heating element 21 in the gap will dissipate throughout the space within the shell 11.

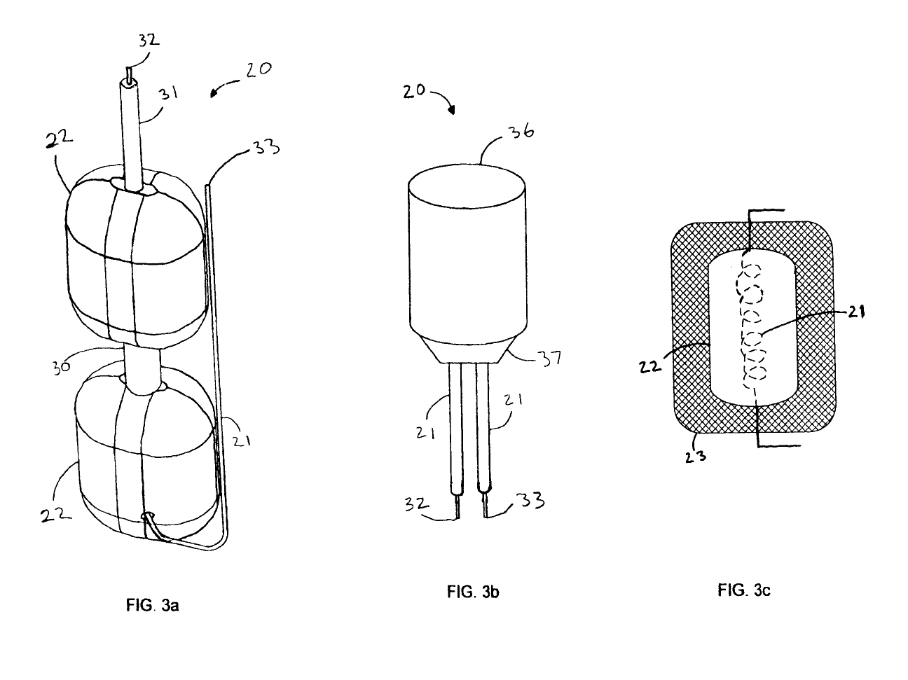

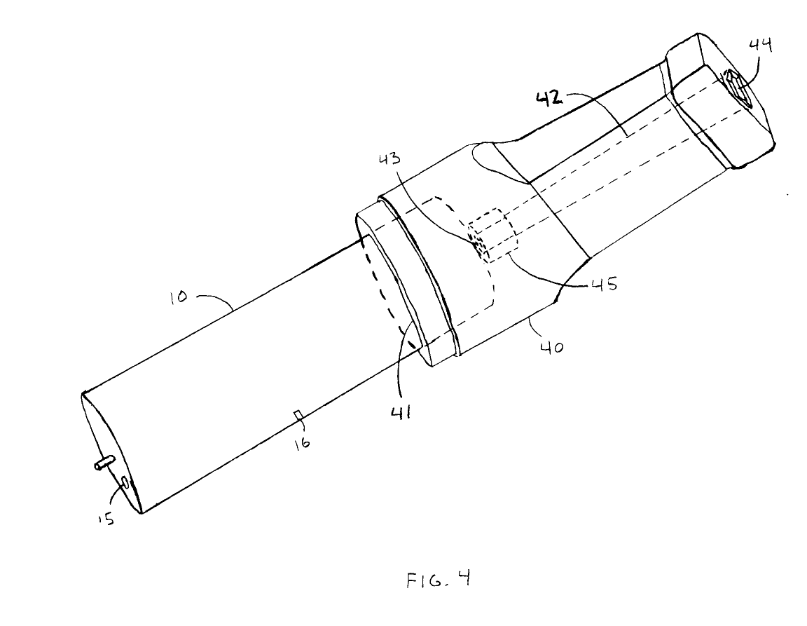

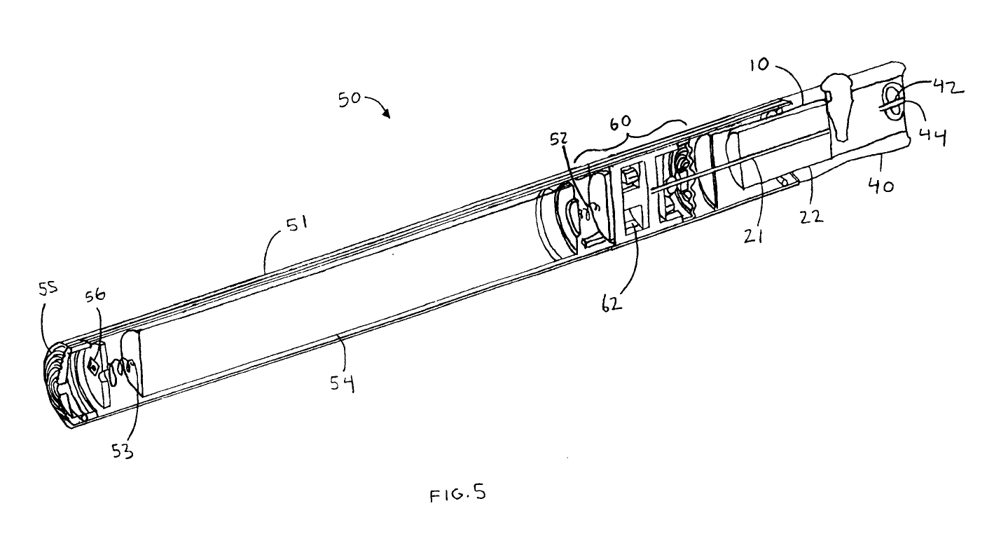

FIGS. 3a-c illustrate different embodiments of the vaporization system 20. FIG. 3a shows the dumbbell embodiment described above. The two reservoirs 22 may be separated by a spacer 30 which maintains exposure of the surface area of each reservoir 22. The spacer 30 may be an insulator, and is preferably the same material as the heat retention structure 23 so as to focus the capillary action of the reservoir 22 toward the center of the heating element 21. The leading end 32 and trailing end 33 of the heating element 21 act as the electrical contacts for passing current and thereby heating the heating element 21. The leading end 32 of the heating element 21 may be covered by wire insulation 31 as it enters the cartridge 10 through the cap 13. The trailing end 33 of the heating element 21 may exit the cartridge 10 through the cap 13 as shown, but preferably meets the base 12. The leading end 32 and the base 12 then make electrical contact with the terminals of the power source upon insertion into the inhalation device.

Referring to FIG. 3b, the heating element 21 comprises two graphite rods connected to opposite polarities and heated to vaporize the solution. A heavier density reservoir 36 stores the solution and provides initial capillary action, while a lighter density reservoir 37 interposed between the heavier density reservoir 36 and the heating element 21 serves as the main wick, having greater capillary action than the heavier density reservoir 36.

Referring to FIG. 3c, the heating element 21 is a wire made of nickel chromium, coiled and encapsulated in a heat retention structure 23. The reservoir 22 is wrapped around the heat retention structure 23.

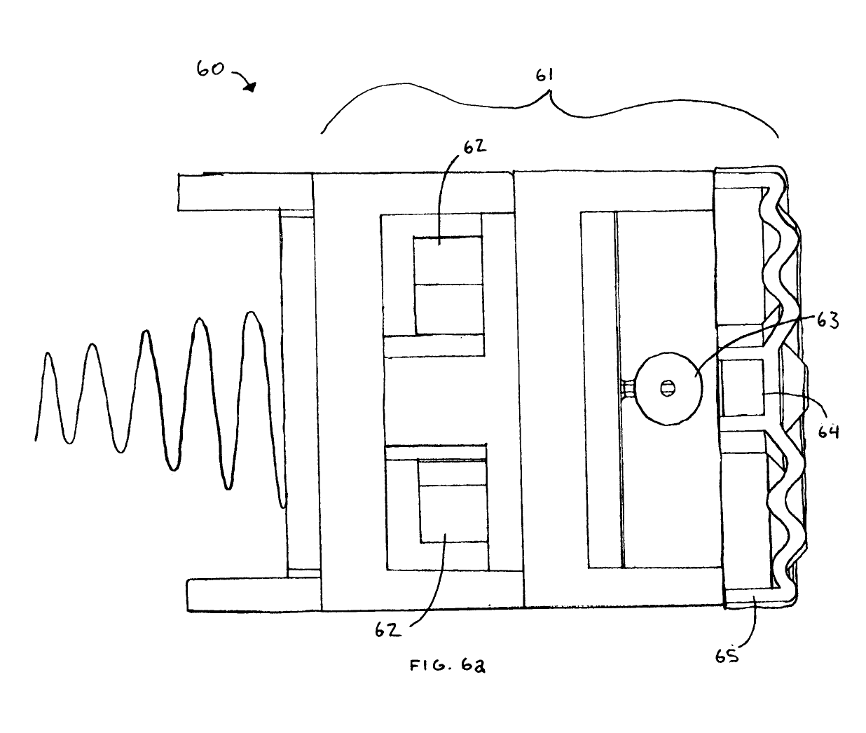

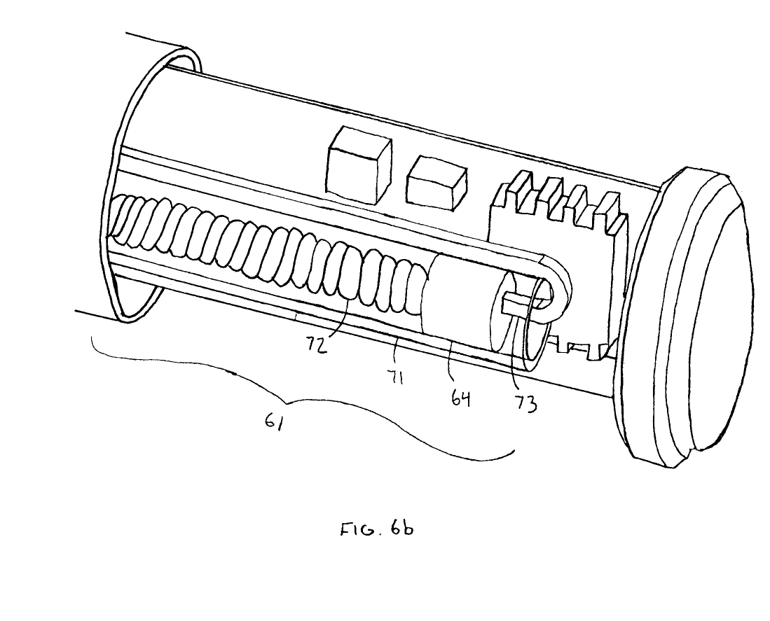

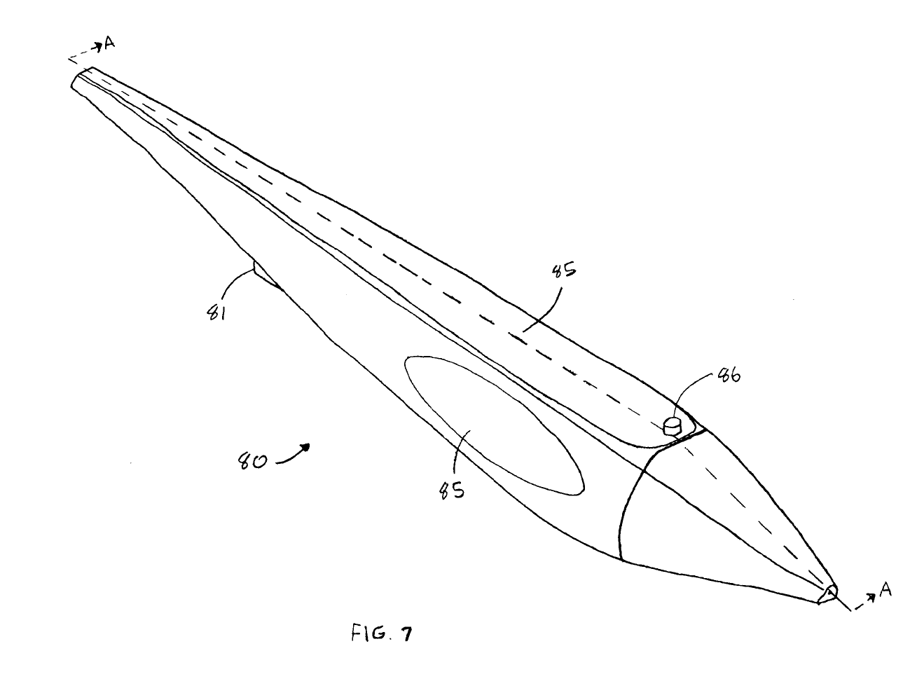

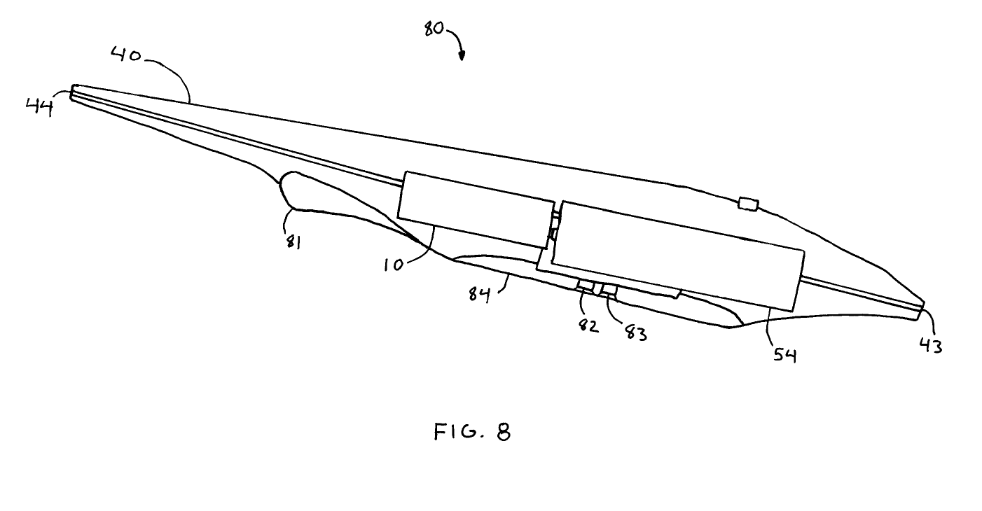

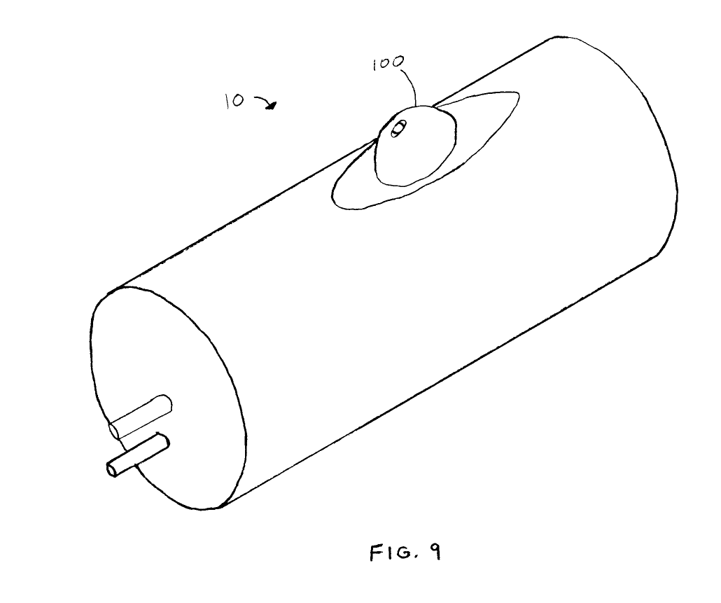

FIG. 4 illustrates the preferred embodiment of the cartridge 10 attached to a mouthpiece 40. The mouthpiece 40 may be wood, ceramic, porcelain, glass, rubber, or plastic, and may be assembled or cast from a mold as a single piece. Preferably, the mouthpiece 40 is vulcanized rubber cast from a mold. The mouthpiece includes an open end 41 which accepts the cartridge 10, an airway 42 extending between an inlet 43 and an outlet 44, and a p-trap 45 surrounding the inlet 43. The p-trap 45 is designed to catch unvaporized droplets of the solution so they do not pass through the airway 42 into the user's mouth. The p-trap 45 may be integrated into the mouthpiece 40 or may be a separable piece which can be replaced if its performance degrades due to use. In the preferred embodiment, the cartridge 10 and mouthpiece 40 are designed to be disposed once the contained solution is fully vaporized. Alternatively, the mouthpiece 40 may be integrated into the inhalation device, as discussed below.

Referring to FIG. 5, in the preferred embodiment the mouthpiece 40 attaches to the cartridge 10, which is then inserted into the inhalation device 50. The inhalation device 50 may be portable or non-portable, but is preferably portable. Preferably, the inhalation device 50 is shaped like a cigarette, having a long, cylindrical case 51 as shown in FIG. 5. The inhalation device comprises electrical terminals 52, 53 which connect to the leading end 32 and trailing end 33 of the heating element 21 when the cartridge 10 is inserted. The electrical terminals 52, 53 pass through a switch 60 and connect to a power source 54. In non-portable embodiments, the power source 54 may be standard mains. In the preferred, portable embodiment, the power source 54 is a battery contained within the case 51. The inhalation device 50 may have an air inlet (not shown) which, upon insertion of the cartridge 10, aligns with the air inlet 15 on the cartridge 10 to facilitate the flow of air through the cartridge 10 into the user's mouth. The end of the inhalation device 50 opposite the mouthpiece 40 may include an ash emulator to give the user the impression of smoking a cigarette. The ash emulator may comprise a cover lens 55 and a light emitting diode 56 which activates in conjunction with the heating element 21. The combination of cover lens 55 and diode 56 resemble the tip of a burning cigarette when lit.

Within the switch 60, a response structure 61 changes state when the user inhales through the mouthpiece 40, and changes state again after a predetermined period passes or when the user stops inhaling. Logic boards 62 detect the state change in the response structure 61 and activate or deactivate the heating element 21. The predetermined period for heating the heating element 21 may be stored in the logic boards 62 and may be based on the desired dosage of vaporized solution in a single inhalation. The logic boards 62 may also store usage information including the number and frequency of inhalations, the amount of solution delivered per inhalation, and power requirements that are specific to the type of solution contained in the cartridge 10. The usage information can be used to enable and disable the device and to indicate to the user when the cartridge 10 should be changed. The logic boards 62 may receive the usage information from the cartridge 10 by communication means such as radio frequency identification (“RFID”) technology incorporated into the logic boards 62 and an RFID chip attached to the cartridge 10.

The response structure 61 can be any switching mechanism that responds to a user inhaling through the mouthpiece 40, such as a reed switch, air pressure sensor, temperature sensor, or condensation sensor, or alternatively can be an external user-actuated switch such as a mechanical push-button or a capacitor-based touch sensor. In the preferred embodiment of the response structure 61, shown in FIG. 6a, a reed switch 63 is in close proximity to a magnet 64, but not close enough to magnetize the reed switch 63 and cause it to close. The magnet 64 is attached to a diaphragm 65. When the user inhales, the diaphragm expands, pushing the magnet 64 toward the reed switch 63 and causing a state change in the reed switch 63, namely closing the contact and alerting the logic boards 62 that the user is inhaling. In an alternate embodiment of the response structure 61, shown in FIG. 6b, a non-magnetic tube 71 contains the magnet 64, a spring 72, and a physical stop 73. The reed switch 63, not shown in FIG. 6b, is positioned beneath the non-magnetic tube 71. In the deactivated state, the spring 72 pushes the magnet 64 against the physical stop 73 and holds it there. When the user inhales, the magnet 64 is pulled by the force of the user's inhalation until it magnetizes the reed switch 63, closing the contact and alerting the logic boards 62 that the user is inhaling.

The inhalation device 50 may include the mouthpiece 40 so that the inhalation device 50 is self-contained except for the insertable cartridge 10. FIGS. 7 and 8 illustrate a simplified, one-piece inhalation device 80 which has a manually-activated power switch 81 that the user presses with a finger or thumb. The power source 54 may be permanently disposed within the one-piece inhalation device 80 and may be rechargeable by way of charging ports 82, 83. The cartridge 10 may be inserted through a cartridge hatch 84 which is integrated into the outer contours of the one-piece inhalation device. The exterior surfaces may be partially or fully covered by a textured material 85 to facilitate gripping the device 80 in the user's hand. An indicator light 86 notifies the user that the power is on and may be a light-emitting diode or other low-cost indicator.

In an alternate embodiment, shown in FIG. 9, the sealed cartridge 10 may comprise a refill port 100 which allows the user to add solution to the fluid reservoir 14 as needed. The refill port 100 is a resealable port that accepts a depositor (not shown) but does not allow air or fluid to escape the cartridge 10. Preferably, the refill port 100 is made of rubber and functions like a standard rubber valve-and-needle inflation mechanism found on recreational inflatable balls.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A device for containing a solution to be vaporized and inhaled, the device comprising:
a) a sealed cartridge insertable into an inhalation device; and
b) a vaporizing system substantially inside the sealed cartridge, the vaporizing system comprising:
i. a heating element that makes electrical contact with a power source in the inhalation device when the sealed cartridge is inserted into the inhalation device; and
ii. at least one reservoir configured to retain the solution and transport the solution toward the heating element when the heating element is activated.

2. The device of claim 1 wherein the vaporizing system further comprises a heat retention structure that encircles a portion of the heating element.

3. The device of claim 1 wherein the sealed cartridge comprises an air intake aperture and a vapor outlet aperture that are sealed before the sealed cartridge is inserted into the inhalation device.

4. The device of claim 3 wherein the air intake aperture and vapor outlet aperture are covered by a material that is punctured to provide airflow through the sealed cartridge when it is inserted into the inhalation device.

5. The device of claim 1 wherein the vaporizing system is dumbbell shaped.

6. The device of claim 1 further comprising a heat-dissipating sleeve in contact with an outer surface of the sealed cartridge.

7. An inhalation device comprising:
a) a power source;
b) an insertable sealed cartridge; and
c) a vaporizing system inside the sealed cartridge, the vaporizing system comprising:
i. a heating element that makes electrical contact with the power source when the sealed cartridge is inserted into the inhalation device; and
ii. at least one reservoir configured to retain the solution and transport the solution toward the heating element when the heating element is activated.

8. The device of claim 7 wherein the sealed cartridge comprises an air intake aperture and a vapor outlet aperture that are sealed before the sealed cartridge is inserted into the inhalation device.

9. The device of claim 8 wherein the air intake aperture and vapor outlet aperture are covered by a material that is punctured to provide airflow through the sealed cartridge when it is inserted into the inhalation device.

10. The device of claim 7 wherein the vaporizing system further comprises a heat retention structure that encircles a portion of the heating element.

11. The device of claim 10 wherein the vaporizing system comprises a plurality of reservoirs and the heat retention structure is a spacer separating at least two of the reservoirs.

12. The device of claim 10 wherein the heat retention structure also encircles at least one reservoir.

13. The device of claim 10 wherein the heat retention structure separates the reservoir from the heating element.

14. A device for containing and vaporizing a solution to be delivered to a user through an inhalation device, the cartridge comprising:
a) a sealed cartridge insertable into the inhalation device, the sealed cartridge comprising an air intake aperture and a vapor outlet aperture that are covered by a material that must be punctured before the solution can be vaporized; and
b) a dumbbell-shaped vaporizing system inside the sealed cartridge, the vaporizing system comprising:
i. a heating element in electrical contact with a power source, the heating element comprising one or more nickel chromium wires;
ii. a cylindrical first reservoir positioned so that the heating element passes through the first reservoir substantially parallel to the first reservoir's axis, the first reservoir being configured to retain the solution and transport the solution toward the heating element when the heating element is activated;
iii. a cylindrical second reservoir positioned so that the heating element passes through the second reservoir substantially parallel to the second reservoir's axis, the second reservoir being separated from the first reservoir by a short distance and configured to retain the solution and transport the solution toward the heating element when the heating element is activated; and
iv. a cylindrical, semi-porous heat retention structure that encircles a portion of the heating element.

15. The device of claim 14 wherein the first and second reservoirs are nickel foam having a thickness of between about 1.7 and 2.2 millimeters, a density of between 320 and 1450 grams per square meter, and pores of between 450 and 800 microns in size.

16. The device of claim 14 wherein the heat retention structure comprises an unglazed ceramic tube.

17. The device of claim 14 wherein the heat retention structure comprises a spacer positioned between the first and second reservoirs.

18. The device of claim 14 wherein the heat retention structure also encircles the first reservoir.

19. The device of claim 18 wherein the heat retention structure also encircles the distance between the first and second reservoirs.

20. The device of claim 14 wherein the heat retention structure separates the first and second reservoirs from the heating element.
